(19) United States
(12) Patent Application Publication
(10) Pub. No.: US 2010/0057091 A1
(43) Pub. Date: Mar. 4, 2010

(54) METHOD AND DEVICE FOR TIGHTENING A SURGICAL CABLE

(76) Inventor: Rogier Oosterom, Utrecht (NL)

Correspondence Address: NIXON & VANDERHYE, PC, 901 NORTH GLEBE ROAD, 11TH FLOOR, ARLINGTON, VA, 22203, US

(21) Appl. No.: 12/522,451

(22) PCT Filed: Dec. 13, 2007
(86) PCT No.: PCT/EP07/10958

§ 371 Date: Nov. 9, 2009

(30) Foreign Application Priority Data
Jan. 10, 2007 (EP) 07000393.4

(51) Int. Cl. A61B 17/58

(52) U.S. Cl. 606/103

(57) ABSTRACT

The invention relates to a method for tying together objects, in particular for fixing hone parts by a surgical cable (10), comprising the steps of laying the surgical cable, having two end parts, around at least part of the objects to be tied together, in particular the bone parts (40) to be fixed, bringing the two end parts together and tying them with a connection, which is slipping when a relatively high force is applied, exerting this relatively high force on the end parts which allows the connection to slip, thereby bringing the cable under a tension required for tying together the objects, in particular for fixing the bone parts. The invention also relates to a tensioning device (1) for exerting the force on the surgical cable.

The invention relates to a method and device for tightening a surgical cable around objects, in particular bone parts in order to tie them together.

In surgery a frequent need arises to internally immobilize bone parts that have been separated due to a trauma or in the course of an operation and need to grow together again, or to keep a bone part at some distance and position with respect to another bone part or an orthopedic device such as a splint, further denoted as fixing bone parts. In treating bone fractures fixing the previously separated bone parts is required for at least the time needed to allow the bones to grow together. Longer periods of time, in many cases even years, may however also be required.

In a known surgical method, a steel cable is wrapped around the bone parts to be fixed, brought under the required tension to secure the parts against relative movement, e.g. under load, and then left in place inside the body at least until the bone parts have grown together and the bone has recovered sufficiently to take up its proper function. The cable may also remain inside the body permanently to avoid a further operation to remove the cable. The cable is tensioned and fixed by guiding its ends from opposite sides through holes in a metal block, tensioning the cable by exerting a drawing force on the ends using a tensioning device, and pinching the metal block such that the holes collapse and fix the cable by a clamping force.

The use of separate fixing devices is inconvenient for a surgeon, who has to handle these tiny devices under the pressure of an operation. Also, devices based on pinching may not work properly in all cases, or may loosen after some time.

The object of the present invention is therefore to provide a method for tying together objects, in particular for fixing bone parts by means of a surgical cable, which method does not suffer from the above mentioned disadvantages and provides easy and secure application of the cable.

The method of the invention thereto comprises the steps of laying the surgical cable, having two end parts around at least part of the objects to be tied together, in particular the bone parts to be fixed, bringing the two end parts together and tying them with a slipping connection, exerting a force on the end parts which exceeds the slip strength of the connection, and thereby bringing the cable under the tension required for tying together the objects, in particular for fixing the bone parts.

The method can in principle be applied to any surgical cable in which a slipping connection can be tied. A particularly suitable cable however comprises ultrahigh molecular weight polyolefin fibers, and ultrahigh molecular weight polyethylene fibers in particular. Fibers of this type are notorious for their difficulty to be fixed by knots, clamps or other means when they are under tension. The essence of the invented method is that this drawback is now used to advantage. In particular, by using this property, a connection is readily provided which is slipping only then when relatively high forces are applied at the free cable ends. As a result of the high strength of the ultrahigh molecular weight polyolefin fibers, and ultrahigh molecular weight polyethylene fibers in particular, such high tensioning forces can be sustained by the cable without fracturing. The connection is preferably made by tying a knot, although the skilled person may easily envisage other types of such slipping connections. According to the invention, after making the slipping connection, e.g. around two or more bone parts, the two free ends of the knotted cable are pulled. When the force applied is high enough, the knot will slip, thereby tightening the cable around the bone parts. In this process, part of the pulling energy brought into the cable ends will be dissipated in the slipping connection. This means that the build up tension in the cable ends will on the average be higher than the average tension in the cable loop around the objects, in particular the bone parts. Preferably, the average tension in the cable ends will exceed the average tension in the cable loop by a factor of 4 to 5, in order to let the connection slip and tense the loop further. Ultrahigh molecular weight polyolefin fibers, and ultrahigh molecular weight polyethylene fibers in particular, have sufficient strength to allow applying such high tensioning forces.

In the method of the invention, the connection is made such that the strength of the cable and the connection exceeds the average tension required to allow the connection to slip, thereby tightening the cable around the object and thus tying together the objects, in particular fixing the bone parts. This will allow to bring the cable under the required tension by pulling the cable ends and letting the connection, or preferably the knot, slip. When the required tension has been reached, the cable ends may be released. In a preferred embodiment of the method, the average tension in the cable around the objects, e.g. due to expansion of the bone parts, is lower than the force which will allow the connection to slip. In this way, the connection will not slip but will actually sustain the tension in the cable around the bone parts, at least for some time. A separate fixing device is in principle not required therefore. The slip strength of the connection depends on a number of factors, including for instance the fibrous structure of the cable, the cable material properties, and, in the preferred case of a knot, its construction. The skilled person can easily adjust slip strength by mere experimentation.

Although the method according to the invention in principle allows tying together objects, in particular fix bone parts, for a prolonged time period, a preferred method includes the step of locking the tensioned cable against the influence of forces acting counter to the exerted force for some period, in case of an operation at least for the post-operative period. Fixing of the cable may be done by guiding the free ends from opposite sides through holes in a metal block, and pinching the metal block such that the holes collapse and fix the cable. Preferably locking the tensioned cable against the influence of forces acting counter to the exerted force is achieved by tying at least one additional knot in the cable ends. The number of knots required depends on the characteristics of each particular case. Additional knots may also be used to adjust the slip strength of the slipping connection, particularly when this connection comprises a knot.

According to the invention the surgical cable is laid around at least part of the objects to be tied together, in particular the bone parts to be fixed. Preferably, the surgical cable is laid around the complete objects to be tied together by forming a loop around the complete objects. Another option is to form a plurality of loops, for example 2, 3 or even 4 or more loops with the surgical cable around the objects to be tied together, in particular the bone parts to be fixed. This offers the advantage of a more secure connection.

Ultrahigh molecular weight polyolefin fibers are known per se, and have an elongate body whose length dimension exceeds the transverse dimensions of width and thickness. The term fibers includes but is not limited to a filament, a multifilament yarn, a tape, a strip, a thread, a staple fiber yarn, and other elongate bodies having a regular or irregular cross-section. Preferably, the ultrahigh molecular weight polyolefin fibers used in the cable have a tensile strength of at least 1.2 GPa, preferably at least 1.8 GPa, and a tensile modulus of at least 40 GPa, preferably at least 60 GPa. Using steel cables for tying objects, and particularly bone parts, together, brings about a number of disadvantages. They are prone to fatigue, leading to fracture of the composing steel fibers, the sharp ends of which stick out into the body. Fracture of the fibers during their application by a surgeon entails the risk of stitching and possible blood contact. Further, steel is a hard material and therefore brings the risk of carving into the bone when tensioned around it. The use of ultrahigh molecular weight polyolefin fibers in the method of the invention does not have these drawbacks, yet offers the desired strength level.

Homopolymers and copolymers of polyethylene and polypropylene are particularly suitable polyolefins for the production of the ultra-high molecular weight polyolefin fibers. The polyolefins may contain small amounts of one or more other polymers, in particular other alkene-1-polymers. A particularly preferred polyolefin comprises ultrahigh molecular weight linear polyethylene, having a weight average molecular weight of at least 400,000 g/mol, more preferably at least 1,000,000, most preferably at least 2,000,000. In the context of this application, linear polyethylene means a polyethylene having less than 1 side chain per 100 C atoms, preferably less than 1 side chain per 300 C atoms.

Preferably polyethylene fibers are used which comprise polyethylene filaments, prepared by a gel spinning process. A suitable gel spinning process is described in for example GB-A-2042414, GB-A-2051667, EP 0205960 A and WO 01/73173 A1, and in “Advanced Fiber Spinning Technology”, Ed. T. Nakajima, Woodhead Publ. Ltd (1994), ISBN 185573 182 7. In short, the gel spinning process comprises preparing a solution of a polyolefin of high intrinsic viscosity, spinning the solution into filaments at a temperature above the dissolving temperature, cooling down the filaments below the gelling temperature, thereby at least partly gelling the filaments, and drawing the filaments before, during and/or after at least partial removal of the solvent.

The method according to the invention may be used for tying any objects together by tensioning a cable around the objects. These fields of applications are less critical than the fixing of bone parts however, and the advantages of the invention and the preferred embodiments thereof in particular manifest themselves in the fixing of bone parts. The method is also useful for connecting bones to artificial elements providing some supporting function, such as a splint for instance.

A particularly preferred cable is a bundle of parallel, twisted or braided fibers of the type described above. The cable may also comprise a tape having the required strength and modulus. The tape may be a single tape or may be in the form of a flat braid of ultrahigh molecular weight polyolefin fibers. Twisting and braiding are commonly applied in cable production and cables obtained by these techniques are applicable in the method according to the invention. In constructing braids and twisted bundles, an efficiency loss usually occurs, which means that the resulting strength of the construction is lower then the average strength of the constituting fibers. In the state of the art, the tension forces in each separate cable required to fix bone parts generally do not exceed 800 N. This is to prevent the cable from cutting through soft bone or bone suffering from osteoporosis for instance, or to prevent crushing of bone fragments. Also, when using steel cables too high tension forces may cause fraying or fracture of the cable. According to the invention, pulling forces on the free cable ends, after making the knot, are preferably higher than 500 N, more preferably higher than 750 N, even more preferably higher than 900 N, and most preferably higher than 1200 N. These are the preferred pulling forces for a single leg. When pulling on a closed loop using such a cable, which can be made by making a knot at the ends of the two free legs, the preferred pulling forces are twice the indicated values, i.e. 1000 N, 1500 N, 1800 N, and 2400 N, respectively. The cable construction and method of connection should be able to sustain such loads. The cable obviously must be adapted to be positioned around the objects, in particular the bone parts, to be fixed. In particular, its length should be sufficient to be laid around the objects, to be tied together by applying a slipping connection, and to be tensioned by pulling the free ends.

In a preferred method according to the invention the force exerted on the end parts of the cable yields a tensile stress in the end parts of at least 650 MPa, more preferably at least 800 MPa, still more preferably at least 1000 MPa, and most preferably at least 1200 MPa. The use of ultrahigh molecular weight polyethylene fibres in particular allows to tension the cable at such high stress levels.

A preferred embodiment of the method according to the invention comprises releasing the tension after tensioning the cable for the first time, adjusting the connection, preferably by adding one or more knots, and subsequently applying a preferably higher tension to the free legs, to allow the adjusted connection to slip, thereby increasing compression force on the objects. More preferably, after releasing the tension again, additional knots are applied to increase knot security, and the legs are cut off at the desired length. The advantage of this embodiment is that the level of compression can easily be adjusted and the security of the connection increased.

The ends of the cable may, when shaped as a bundle of fibers, be treated to prevent unraveling or splitting of the bundle. The ends may for instance be glued together with a suitable substance, or they may be melted together or otherwise be prevented from unraveling. A cable end may also be formed into an eye by splicing the end back into the fiber bundle. In this case eyes form the end parts of the cable.

According to the invention, the cable is preferably positioned around the objects, in particular the bone parts, following a trajectory that is stable when the cable is tensioned. This prevents the cable from moving to a shorter trajectory, leading to loss of tension in the cable and consequently loss of fixing. Generally this will be the shortest trajectory at a certain position along the objects or bone parts. Alternatively the cable along the trajectory may be prevented from sliding to a shorter one by natural obstacles as bone processes or artificial fixing devices or protrusions applied to the bone parts.

In the method of the invention a slipping connection is made in the cable, preferably by tying one or more knots. Suitable knots for this purpose are in principle elementary simple knots. Examples of suitable knots are a flat knot, a loop knot, a surgeon knot, a water knot, a tape knot, a double figure eight knot and a double overhand bend, or any combination thereof. These knots may also be applied as additional knot, but preferably a two flat half or flat overhand knots are used for this purpose. The person skilled in the art may easily select other suitable knots, for example the so-called double ring hitch, the Kellig hitch, or Prusik and Klemheist knots. These and other knots, and methods to make them can be found in the ‘Handbook of knots’, (Dorling Kindersley Book, London 1998; ISBN 0751305367), and in “The Ashley book of knots’ (Faber and Faber Ltd, London 1990; ISBN 057109659x).

After the slipping connection has been made in the cable, the cable ends are pulled with a certain tensile force to tighten the cable loop around the objects, in particular the bone parts. Preferably a tensioning device is used for this purpose, which will be described below. In all embodiments disclosed the tensioning action is continued until the required tension in the cable is achieved. Subsequently, the tensioned cable must be locked against the influence of forces acting counter to the exerted force.

The invention also relates to a tensioning device for a surgical cable. The tensioning device is to be used in conjunction with the above described method. Known tensioning devices cannot be used since the forces required to tension the cable by far exceed their load bearing capacity. In US 2004/0127907 at p. 3, [0051] it is said that tension force in cable normally is between about 5 to about 800 N. If the tension force in the cable is too high, damaging of the objects to be tied together may take place. For that reason the known devices are designed to be restricted to that tension. Because devices are manually operable it is not possible to apply the high tension, also devices would not be stiff and/or strong enough to sustain the required tension levels. Some devices even have special mechanisms to put an upper limit to the tension that may be applied in the cable. In the method according to the invention however the level of tension force at the ends of the cable may exceed 900 N, preferably exceeds 1200 N, more preferably exceeds 1400 N, even more preferably exceeds 1600 N and most preferably exceeds 2000 N. These tension levels preferably apply in case of a braid construction made of Dyneema Purity® or Spectra with a diameter of between 0.7 and 1.4 mm. In case a closed loop made of the same cable is tensioned, by placing a non-slipping knot at the end of the two legs, the tensioning device should be able to support tension levels exceeding at least 1000 N, more preferably exceeding 1500 N, even more preferably exceeding 1800 N, and most preferably exceeding 2400 N. The tensioning device according to the invention comprises a restraining body adapted to restrain the surgical cable to be laid around at least part of the objects to be tied together, and an adjusting mechanism adapted to cooperate with the restraining body to change the tensile force applied to the surgical cable, with the proviso that the device is adapted to allow the surgical cable to be tensioned by the restraining body at a tension of at least 900 N. Preferably the tensioning device is adapted to allow the surgical cable to be tensioned by the restraining body at a tension of at least 900 N, even more preferably at least 1200 N, even more preferably at least 1500 N, still more preferably at least 1800 N and most preferably at least 2400 N.

The device is adapted to allow the cable to be tensioned, means that the apparatus will not be damaged, like breaking or bending, or that no restrictions are in the apparatus to raise the level of tension force. Preferably the device is operated by a an electrical motor.

The tensioning device is adapted for holding the end parts of the cable. In case a cable having end parts in the form of an eye is applied the device may comprise two hooks or similar that each can hook to one of the eyes of the cable and be provided with means to draw the hooks to one another. Such means can comprise a mechanism as used in turn buckle, a worm wheel and driving screw combination or two cooperating 45 [deg.] tooth wheels rotating around mutually perpendicular axes. These tensioning devices can be connected to the cable in such a way that only a drawing force is exerted on the cable, resulting in its shortening and tensioning but also in such a way that, instead of or next to the drawing force, also a twisting force is exerted on the cable, also resulting in further tensioning the cable.

A preferred embodiment of the tensioning device according to the invention has a restraining body comprising an adjustable frame, provided with at least two guiding means in opposite corners of the frame, between which means a surgical cable can be tightened, the adjusting mechanism being adapted to change the relative distance between said corners of the frame. The adjusting mechanism may be mechanical, in which case it preferably comprises a rotatable member, such as a screw. Such a device is easily manipulated, and allows to adjust the average tension in the cable ends with the precision required. Another preferred adjusting mechanism comprises a hydraulic pump.

In order to be able to preset a certain tension in the cable the tensioning device is preferably equipped with measuring means for the applied tension. Such means are known per se and any known means to measure forces may be used.

The invention will now be further explained by the following figures, without however being limited thereto. Herein:

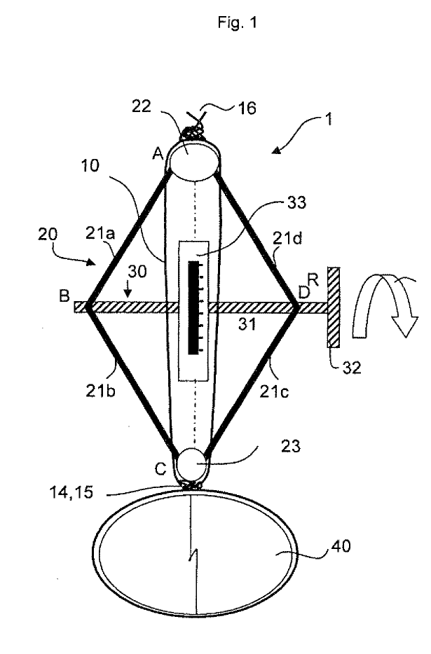

FIG. 1 schematically represents a tensioning device according to the invention; and

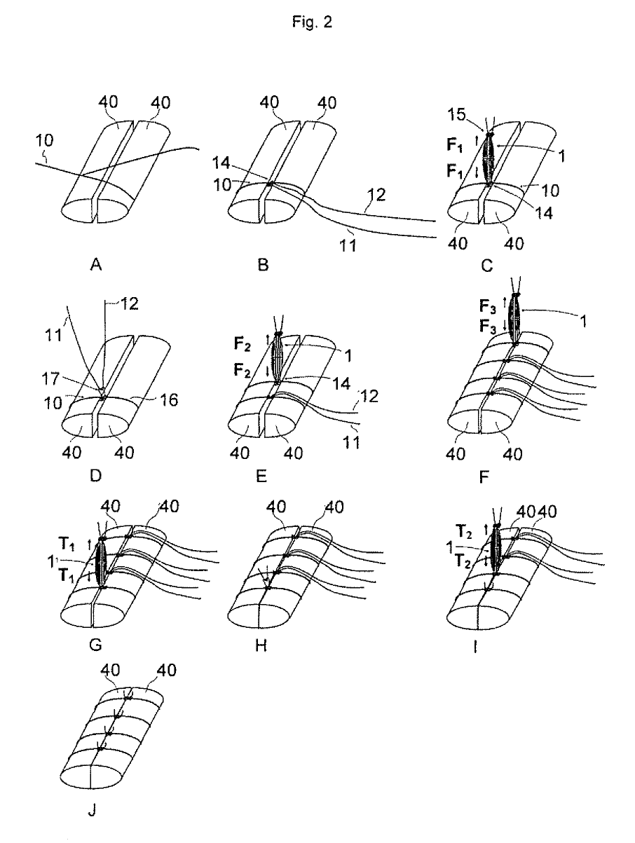

FIG. 2 schematically represents a possible sequence of steps of the method according to the invention.

With reference to FIG. 2, in the method according to the invention a surgical cable 10, is laid around bone parts 40 in order to fix them (FIG. 2A). Bone parts 40 may for instance comprise a cut bone piece, e.g. a sternum for the purpose of an open-heart surgery. Surgical cable 10 consists of a braid of ultrahigh molecular weight polyethylene fibers (Dyneema® SK75 yarn, 1760 dTex). Cable 10 has two end parts 11 and 12, which may consist of single cable, but which may also be formed by folded back portions of cable 10, forming a loop. A first surgical cable 10 is laid around or through the bone parts 40 (FIG. 2A), for instance using a needle, and a first slipping connection in the form of a knot 14 is placed in this cable by bringing the two end parts (11, 12) together and tying them (FIG. 2B). As shown in FIG. 2C, the remaining legs (11, 12) from the first knot 14 are then positioned around a tensioning device 1, in this case each with an additional loop around the tensioning device and tightly fixed with a non-slipping knot 15, for instance by hand-force. A tensioning force F1 is then applied to increase compression onto the bone pieces 40. The manner in which the tensioning force may be applied by the tensioning device 1 is described further below.

According to the invention the force F1 exerted on the end parts (11, 12) exceeds the force to allow slip of the knot (14), using a tensioning device 1, which is able to sustain an average tension in the cable of at least 500 N, without substantial loss of tension. With the phrase “without substantial loss of tension” is meant that the tensioning device 1 is dimensioned such that the tension in cable 10 remains substantially equal to the required (preset) tension, at least during the process of tying together the objects, in particular the bone parts 40. In this process of tightening, the build up tension in the cable ends (11, 12) will on average be higher than the average tension in the cable loop 16 around bone parts 40. According to a preferred embodiment, the tensioning force is then released, the legs (11, 12) are cut off just under the non-slipping knot 15 and the tensioning device 1 is then removed, as shown in FIG. 2D. Enough length in both legs (11, 12) will be available thereafter, if they are positioned around the tensioning device with an additional loop. After having tightened the cable loop 16 around bone parts 40, the tensioned cable is locked against the influence of forces acting counter to the exerted force by tying at least one additional knot 17, for instance by hand-force.

A second cable 10 may then be positioned around or through the bone parts 40, e.g. using a needle, and a first slipping knot 14 in this cable is placed. Again, the remaining legs (11, 12) from the first knot 14 in this second cable 10 are positioned around the tensioning device 1, again with an additional loop, and tightly fixed with a non-slipping knot 15, for instance by hand-force. A tensioning force F2 is then applied to increase compression onto the bone pieces 40 (see FIG. 2E). Positioning of the second cable (and further cables) may influence the tension in the first cable (and other previously applied cables). For instance the tension in the first cable may drop when the second cable is applied. When this is undesirable, the first cable may be tensioned further after having applied the second cable in order to increase its tension to the desired level. Preferably, a fixating device is then temporarily used. It is also possible to initially overstress the first cable, whereby the tension in the first cable will drop to the desired level upon applying the second cable.

Referring to FIG. 2F, in the embodiment shown, a total number of 4 cables are applied using this approach to fix the bone pieces 40. During the tensioning procedure bone parts 40 will gradually come closer to each other (compare to FIG. 2B for instance). The applied tensioning forces F1, F2, F3 may be equal to each other. It is also possible that they mutually differ, for instance that F1>F2>F3.

After the 4 cables have been tensioned and a second knot has been placed for each cable, a possible second tensioning procedure can be added, as demonstrated in FIG. 2G. A similar approach as in FIG. 2C is used, although it may not be possible to apply an additional loop around the tensioning device due to the limited length of both legs due to the first tensioning procedure. The remaining legs from the second knot in this cable are thus positioned around a tensioning device 1 and tightly fixed with a non-slipping knot, for instance by hand-force. A tensioning force T1 is then applied to increase compression onto the bone parts 40.

As shown in FIG. 2H, the bone parts may touch during the tensioning procedure and will be compressed (compare to FIG. 2G). The tensioning force is released and the tensioning device has been removed. By cutting off the legs just under the non-slipping knot, enough length in both legs will be available to apply final additional knots in this cable, for post-operative security reasons, tightly fixed by hand-force for instance.

As shown in FIG. 2I, the second cable with the additional knot will be tensioned using a similar approach as shown in FIGS. 2G and 2H, including the additional knot for post-operative security.

Finally, as shown in FIG. 2J, all cables will be tensioned using the approach of FIGS. 2G and 2H, and the bone parts will obtain the preferred compression and additional knots may be added for post-operative security.

With reference to FIG. 1 a tensioning device 1 is shown which is preferably used in connection with the above described method, in which a surgical cable 10 is laid around bone parts 40, and provided with a slipping connection (14, 15). The device comprises a restraining body 20 adapted to restrain a portion of the surgical cable 10. Restraining body 20 comprises an adjustable frame 21, consisting of 4 frame members 21a, 21b, 21c, and 21d, Frame members 21a to 21d are mutually connected through 4 pivots A, B, C and D. Frame 21 is further provided with two guiding means (22, 23) in opposite corners A and C of frame 21, between which a surgical cable 10 can be tightened, e.g. by using a non-slipping knot 16 around guiding means 22. Tensioning device 1 is further equipped with an adjusting mechanism 30 being adapted to change the relative distance between corners A and C of frame 1. As shown in FIG. 1, a simple arrangement consists of a rotating screw 31 and handle 32. Other arrangements, such as a hydraulic pump, may also be used. By turning the handle 32 in direction R, the screw will rotate and alter the distance between corners B and D. Since frame members 21a to 21d are stiff, this process also alters the distance between corners A and C. For instance the distance between pivots A and C can be increased by decreasing the distance between pivots B and D. Such an increase of the distance between A and C actually brings the cable 10 under increased tension. Essential to the invention is that the tensioning device is adapted such that it can sustain an average tension in the cable of at least 1000 N, since the tension is applied on both legs, without substantial loss of tension, in particular in case a braid construction made of Dyneema Purity® or Spectra with a diameter of between 0.7 and 1.4 mm is used. In order to monitor the average tension in cable 10, tensioning device 1 is equipped with measuring means 33 for the applied tension. Any suitable means known in the art to measure forces may be used as such. When used for tensioning a surgical cable tensioning device 1 should be made of material that is easily cleanable, and that can be readily sterilized.

1. Method for tying together objects, in particular for fixing bone parts by a surgical cable, comprising the steps of laying the surgical cable, having two end parts, around at least part of the objects to be tied together, in particular the bone parts to be fixed, bringing the two end parts together and tying them with a slipping connection, exerting a force on the end parts which exceeds the slip strength of the connection, thereby bringing the cable under a tension required for tying together the objects, in particular for fixing the bone parts.

2. Method according to claim 1, wherein the force exerted on the cable ends to increase compression on the objects is 2 to 10 times higher than the force in the cable around the objects, more preferably 4 to 5 times higher.

3. Method according to claim 1, wherein the slipping connection comprises a knot.

4. Method according to claim 1, wherein the connection is made such that the slip strength of the connection exceeds the average tension required for tying together the objects, in particular for fixing the bone parts, by 20% of the tension required.

5. Method according to claim 1, including the step of locking the tensioned cable against the influence of forces acting counter to the exerted force.

6. Method according to claim 5, wherein locking the tensioned cable against the influence of forces acting counter to the exerted force is achieved by tying at least one additional knot.

7. Method according to claim 1, wherein the cable comprises ultrahigh molecular weight polyethylene fibers.

8. Method according to claim 1, wherein the exerted force is a tensile force, and the force is exerted by a tensioning device.

9. Method according to claim 1, wherein the exerted force yields a tensile stress in the end parts of at least 650 MPa.

10. A tensioning device for a surgical cable comprising:
a restraining body adapted to restrain the surgical cable to be laid around at least part of the objects to be tied together;
an adjusting mechanism adapted to cooperate with the restraining body to change the tensile force applied to the surgical cable;
whereby the device is adapted to allow the surgical cable to be tensioned by the restraining body at a tension of at least 1000 N.

11. A tensioning device according to claim 10, whereby the device and the surgical cable are adapted to allow the surgical cable to be tensioned by the restraining body at a tension of at least 900 N, more preferably at least 1000 N, still more preferably at least 1200 N, and most preferably at least 2400 N.

12. A tensioning device according to claim 10 further comprising measuring means for the applied tension.

13. A tensioning device according to claim 9, wherein the restraining body comprises an adjustable frame, provided with at least two guiding means in opposite corners of the frame, between which means a surgical cable can be tightened, the adjusting mechanism being adapted to change the relative distance between said corners of the frame, thereby increasing or decreasing the tension in the cable, if the relative distance is increased or decreased, respectively.

14. Surgical cable prepared for application in a method according to claim 1, or for application in the tensioning device.
